(19) United States
(12) Patent Application Publication
(10) Pub. No.: US 2018/0161569 A1
(43) Pub. Date: Jun. 14, 2018

(54) Scalp-Mounted Sensory Prosthesis and Method of Use

(71) Applicant: Liv Maria Kelley, Durham, NC (US)

(72) Inventor: Liv Maria Kelley, Durham, NC (US)

(21) Appl. No.: 15/373,462

(22) Filed: Dec. 9, 2016

(51) Int. Cl. A61N 1/04; A61N 1/36; A61H 23/00; A61H 7/00

(52) U.S. Cl. CPC A61N 1/0476; A61N 1/36014; A61H 2205/021; A61H 7/001; A61H 23/00

(57) ABSTRACT

A sensory prosthesis is disclosed herein with a data recording device configured to record sensory information around a user, with the data recording device connected to a plurality of wires. The wires send signals from a computer positioned on or proximate to the user that translates sensory information from a camera, olfactory sensor, auditory recording device, or sensors attached to areas of the body with limited tactile function into electrical signals. The computer is connected to a power source configured to send signals from the computer to stimulators that are attached to the user's scalp. Corresponding systems and methods also are disclosed.

### BACKGROUND

General sensory prostheses like the devices created by American neuroscientist Dr. Paul Bach-y-Rita can be difficult to conceal and unwieldy, or can interfere with eating or drinking. For example, the device described in U.S. Utility Pat. No. 6,430,450, is unwieldy and difficult to use because it contains many of the basic principles of a visual prosthesis including the translation of visual information into electrical signaling, but is used in the mouth, which requires the wearer to remove the visual support device while eating or talking. There is precedent for devices which are more sleek, portable, and easy to conceal, but unfortunately, these devices are not generally geared towards acting as sensory prosthesis. One such device is disclosed in US Patent Application Publication US20050119702A1, but this device has limited capability to communicate pictorial information due to a limited number of electrodes. This device can only serve a sensory feedback function, while a device with more electrodes or stimulators would be more sensitive in order to convey more visual information to the user.

It would be useful to develop/improve the placement of electrodes or tactile stimuli, power source, and sensory apparatus on a sensory prosthesis. A differently designed prosthesis may solve the cumbersome aspects of previous designs.

### SUMMARY

One embodiment described herein is a sensory prosthesis comprising a data recording device configured to record visual, olfactory, sound, and rerouted tactile data types proximate a user, a plurality of stimulators attached to the user's scalp, and a computer positioned on or proximate to the user. The computer is configured to translate the recorded sensory information into signals using a program. The prosthesis also includes a plurality of wires configured to send signals from the computer to the stimulators, and a power source is configured to power the sensing apparatus and the computer.

In embodiments, the data recording device is configured to be removably mounted to the user's skin. In some cases, the plurality of stimulators and/or the plurality of wires are waterproofed. In embodiments, the plurality of wires are disguised.

Certain embodiments of the visual prosthesis comprise stimulators that are tactile or electrical nodes. In embodiments, the plurality of stimulators are microelectrodes, but the stimulator can also be a vibrating, heating, cooling, or pressure-based tactile stimulator.

In some embodiments, the computer comprises a programmed computer chip. Frequently, the power source is a portable and rechargeable battery.

Optionally, the data recording device attaches to the user's clothing. In some embodiments, the data recording device is attached magnetically so as not to fall over. In some cases, the wire grid portion of the prosthesis is supported by a wire hooked over the user's ears. Frequently, the electrodes are laid against the scalp to receive visual information. In embodiments, wire structure is made up of separate segments that can be assembled on the user's scalp.

In further embodiments, the data comprises visual images and a visual prosthesis with waterproofed stimulators and wires. In other embodiments data is tactile information from sensors mounted on some part of the body, olfactory information from an electronic nose or olfactory sensor, or auditory information from an audio recording device.

### BRIEF DESCRIPTION OF THE DRAWINGS

FIG. 1 shows the side view of a first embodiment of a visual prosthesis.

FIG. 2 is a front view of the embodiment shown in FIG. 1.

FIG. 3 shows a back view of the embodiment shown in FIG. 1.

FIG. 4A shows a camera mounted on a necklace.

FIG. 4B shows a camera adhered to a user's body.

FIG. 4C shows a camera adhered to the user's clothing.

FIG. 4D shows multiple cameras adhered to the user's clothing.

FIG. 5 depicts a second embodiment, demonstrating that a lower number of electrodes can be used to collect information.

FIG. 6 shows a third embodiment with an alternate wiring pattern, but similar structures.

FIG. 7 shows a fourth embodiment with an alternate wiring pattern, but similar structures.

FIG. 8 shows a chart detailing a computer program that can be used to convert images into signals in the electrodes or stimulus nodes.

FIG. 9 demonstrates an embodiment in which the elastic forms a different pattern that may be more suitable to some customers' aesthetic choices.

FIG. 10A shows the front view of the device with the olfactory sensor or electronic nose being attached and used to collect olfactory data.

FIG. 10B shows the programming process necessary to process the olfactory data into electrical or tactile information on the scalp.

FIG. 10C shows the front view of the device with the auditory recorder being attached and used to collect auditory data.

FIG. 10D shows the programming process necessary to process the auditory data into electrical or tactile information on the scalp.

FIG. 10E shows the front view of the device with the tactile sensors attached with the help of a longer wire. This tactile sensor is used to collect tactile information from areas of the body lacking tactile capability.

FIG. 10F shows the programming process necessary to process tactile data into electrical or tactile signals on the scalp.

### DETAILED DESCRIPTION

The embodiments described herein comprise sensory prostheses that substitute a different sense or a tactile sense representing certain kinds of information for lost visual, auditory, smell, or feeling capability. However, in embodiments the device extends to cover prostheses relating to olfactory, auditory, and tactile sensory substitution. The visual version of this device works through a form of body camera (13) connected to a chip and to stimulation nodes attached in a web and laid against the skin of the scalp. The visual information from the camera is translated by the chip (20) and delivered to the different nodes (3) on the scalp. These nodes vibrate, push against, or pulse through the scalp in the pattern of the picture the camera shows at any given time. Through these electrical or tactile signals the user can obtain visual information they may not have access to otherwise. Other embodiments described later perform the same function, but using different sensory recording devices and data types.

This visual prosthesis is intended to provide visual information to those visually impaired or to those hoping to supplement their vision by collecting visual information in a camera, processing it using a suitable programming language, such as, for example, the programming language C, and activating small electrodes or stimulators (3) on the scalp to provide an electrical or tactile sensation that creates a detectable “image” of the surrounding area.

As used herein, the term “visual information,” refers to pixels of information representing surroundings that can be translated into electrical or tactile feedback.

As used herein, the term “sensory prosthesis” refers to a device that collects sensory information from surroundings, and translates that into electrical or tactile feedback.

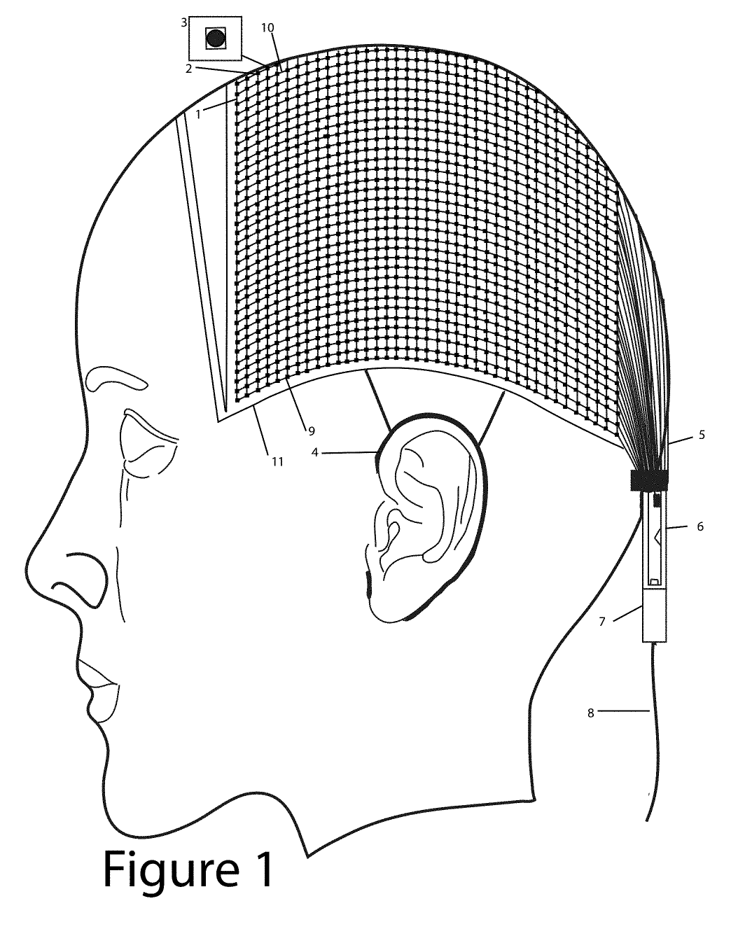

FIG. 1 shows a side view of the visual version of this prosthesis comprising a mesh of wires creating a grid of electrodes that will be laid flush against the scalp. In embodiments, sections of hair are shaved for this process. The sections are shaved in the areas where the electrodes will be placed. The grid of wires attaches a wire (10) to each side of a stimulator (3). A plurality of stimulators (3) are shown in the Figure, and an enlarged view of a single stimulator (3) is shown at the top of the Figure. Each wire (10) then connects to a central computer chip (6) that delivers electrical or tactile signals to the stimulators (3). This chip (6) combined with the program (18) comprises the computer.

Stimulators (3) are secured onto the head using wire portions (1) that attach to elastic lined sides (11) of the grid and to the wire that wraps around the wearer's ears (4) and hold the electrode grid against the user's head. In embodiments, the wires (10) and/or the wire portions (4) hooked or looped around the ears are coated with rubber or plastic for the comfort of the user. Wires (10) from this device have connectors (2) to attach to each stimulator (3) to wires (10) in a crisscross pattern. The wire connectors (2) in some versions may be waterproofed. The stimulators (3) themselves optionally may be waterproofed. One non-limiting example of a waterproofing method involves applying a coating comprising a styrene polymeric film cast from an organic solvent. The result of these processes is that the entire cap structure made by the wires, electrodes, and connectors may be waterproofed so that the user could later get their scalp and hair wet without disturbing the functioning of the device.

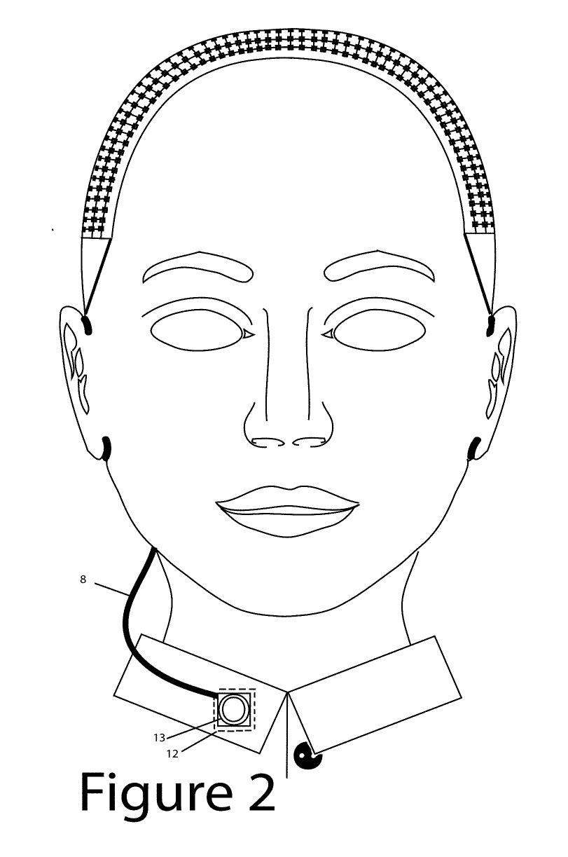

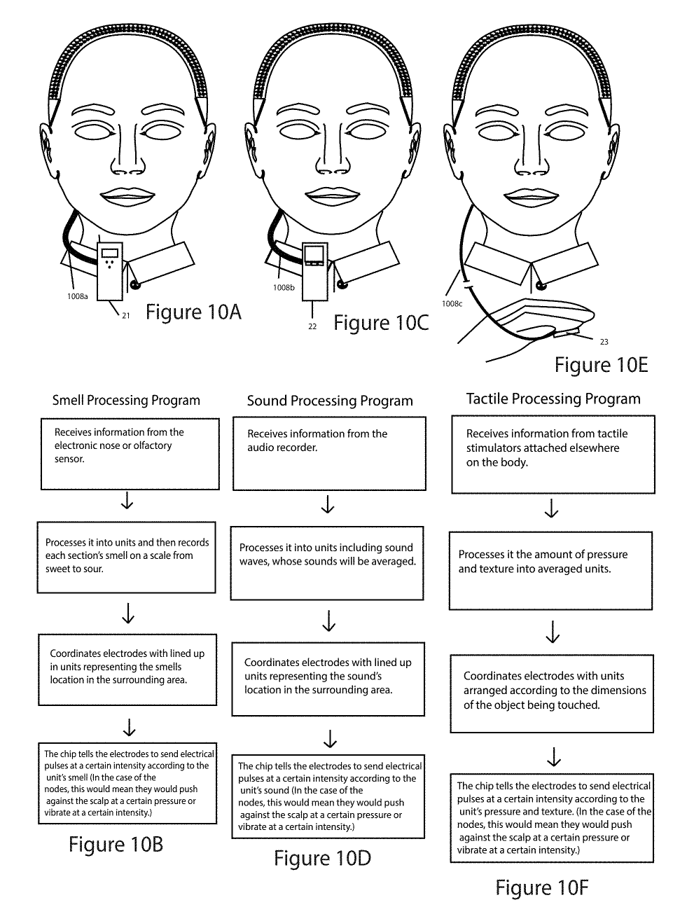

To aid in an understanding of the embodiments disclosed herein, a visual prosthesis is depicted in FIG. 2. However, it is to be understood that the recording device also or optionally can record olfactory, sound, and/or rerouted tactile data and the stimulators also or alternatively convey olfactory, sound, and/or rerouted tactile information as further described in FIG. 10.

Referring to FIGS. 1-3, the visual prosthesis version of this device includes a camera (13), which, for example, can be positioned on the user's collar using a magnet (12) and connected to the computer chip (6) by a wire (8). FIG. 2 demonstrates how elastic (11) and rubber wire (4) secures the electrodes or stimulators (3) and their connectors (2) to the wires (10) in the grid that connect to the edge wires (1). It also shows how all of these wires (10) use connectors (2) to attach to an encased computer chip (6) and portable power source, or battery (7), that in turn connects to the camera wire (8) leading to the camera (13).

The stimulators (3) can be electrodes, or tactile stimulators that apply pressure, heat, or vibration. One example of a suitable electrode is described in U.S. Pat. No. 3,612,061A, “Flexible cutaneous electrode matrix.” This non-limiting example of a suitable electrode involves encasing pressure-based skin-contacting electrodes with elastic material. The elastic material will help to waterproof the electrodes and the skin-contacting aspect helps the patient avoid a partial-implantation process. In the embodiment shown in FIG. 1, unlike the disclosure of U.S. Pat. No. 3,612,061A, each electrode is separated and attached to adjacent electrodes by wires. In embodiments, these electrodes are about 0.1 mm to about 3 mm in width and about 0.1 mm to about 3 mm in height. This configuration allows the patient's hair to cover the electrode system. In some cases, about 4 to about 2000 electrodes are used, or about 30 to about 1500 electrodes or about 200 to about 1000 electrodes.

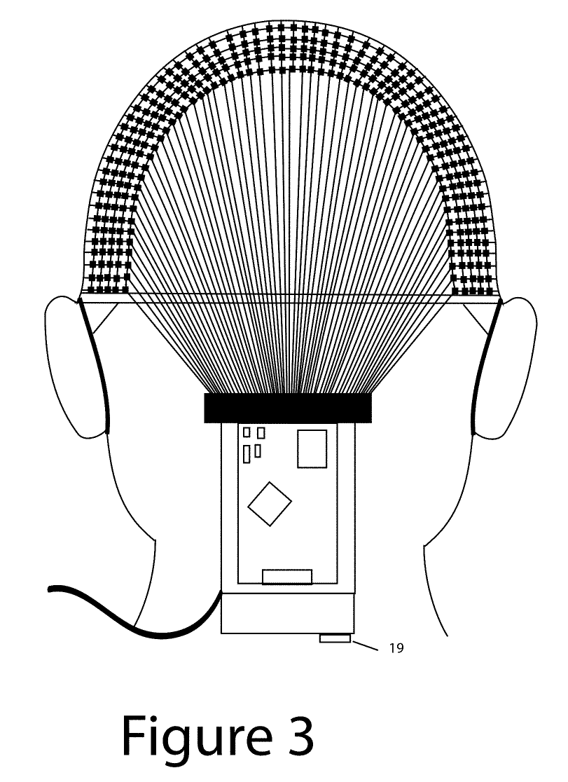

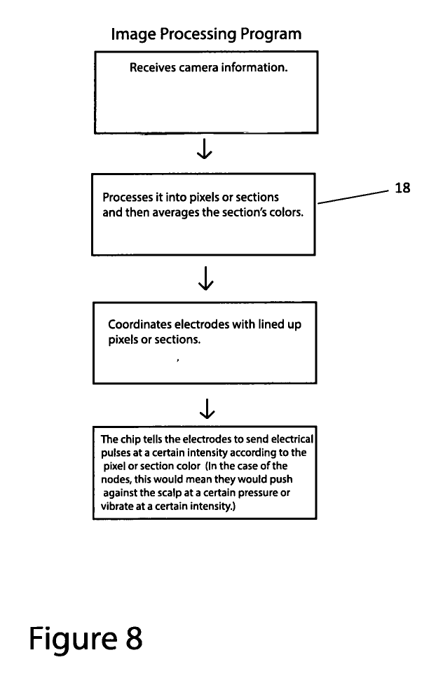

FIG. 3 depicts the back view of the device. The wires (10) gather (5) and attach to the encased chip (6). The chip then connects to the wire leading to the camera (8) with the help of another waterproof connector (2). The chip (6) executes a C-based computer program (18), or another suitable program that can be designed by someone with experience in the art to process the image most recently attained by the camera (13) and translate it into electrical or tactile signals that can be sent to the electrodes or stimulators. In one non-limiting embodiment, the chip (6) executes a program that involves receiving camera information, processes it into pixels, coordinates the pixel location with each electrode, and then makes the stimulators (3) vibrate, press, or give small tactile electric signals at different strengths in order to communicate color value and depth (18). FIG. 8 describes in detail what that process involves and how it allows the device to function.

FIG. 2 also displays a rechargeable battery (7) connected to the end of the chip (6). This battery (7) can be waterproofed using the same methods as can be applied to the electrodes and is attached using wires and waterproof connectors to the chip and the camera, shown in FIG. 3. The battery may have a watertight screw-cap lid (19) over a port through which it can connect to a charging cable that can plug into a computer or a wall. This extra provision can be added because the battery is vital to the performance of the rest of the device, and its continued functioning is important.

FIG. 3 shows the camera (13), and the mechanism or support (13) through which the camera (13) can attach to clothing (12) or jewelry (14). The camera (13) can be a micro video camera or any other suitable camera. The camera (13) optionally can be waterproofed in the same manner as the electrodes and battery. It can be configured to also be removed along with the wire that connects it to the battery (7) and chip (6) prior to activities involving water, such as showering. The setup shown in FIG. 1 attaches the camera (13) to stiff shirt collars using two square magnets (12). In embodiments, one of these magnets can be glued to the back of the camera, and the other can be placed on the inner side of the fabric. This configuration enables the apparatus to be removably connected to the clothing so that the user can comfortably change clothing or shift the device's position without trouble. While this Is designed for a shirt collar, it could also theoretically attach to belts, bracelets, or other rigid garments. It is currently made this way so the camera does not fall forward throughout the user's day.

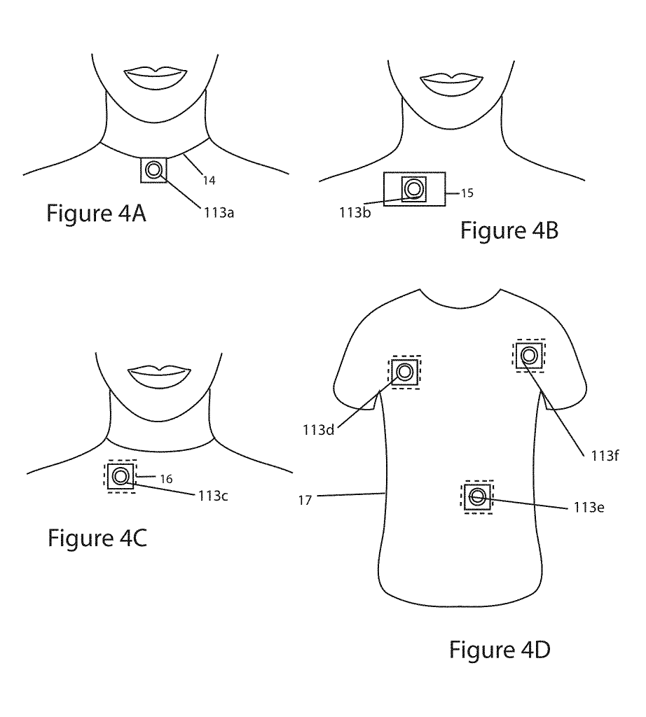

FIGS. 4A-4D show various ways that the camera (13) can be attached to the wearer. FIG. 4A shows the camera (113) on a necklace (14). The camera can be removably or permanently mounted to a base on a chain, string, or strap (14) that is placed around the wearer's neck to increase the aesthetic of the device and the comfort of the wearer. FIG. 4B illustrates a camera (213) stuck to the person's skin using an adhesive (15), such as silicone tape or another suitable material in order to allow the wearer to use different kinds of garments. The camera (113b) can also be secured using double sided acrylic fashion tape (16) or there can also be multiple cameras be secured in various places on the clothing (17). This modification allows the user to attach the camera to a belt or other stiff section of clothing in order to allow the wearer to don different kinds of clothing without having to attach the camera directly to their skin. FIG. 4C shows a camera (113c) attached to a user's clothing near the neckline. This version allows those using the device to use a supportive backing (16). This can be any backing that can keep the camera upright on multiple garments. It provides another option for those who don't like the feeling of the silicone on their skin, but do not have any garments with stiff areas. FIG. 4D shows an embodiment that employs three cameras (113d), (113e), and (113f) which can be adjusted to take photos and/or video in different directions. In the embodiment shown, all of the cameras are attached to the user's shirt, although one or both of the cameras could be mounted in the manner shown in FIG. 4A and/or FIG. 4B. One or more of the cameras could be pointed at the ground to help those with trouble with vision look for obstacles near the ground that may cause the user to trip.

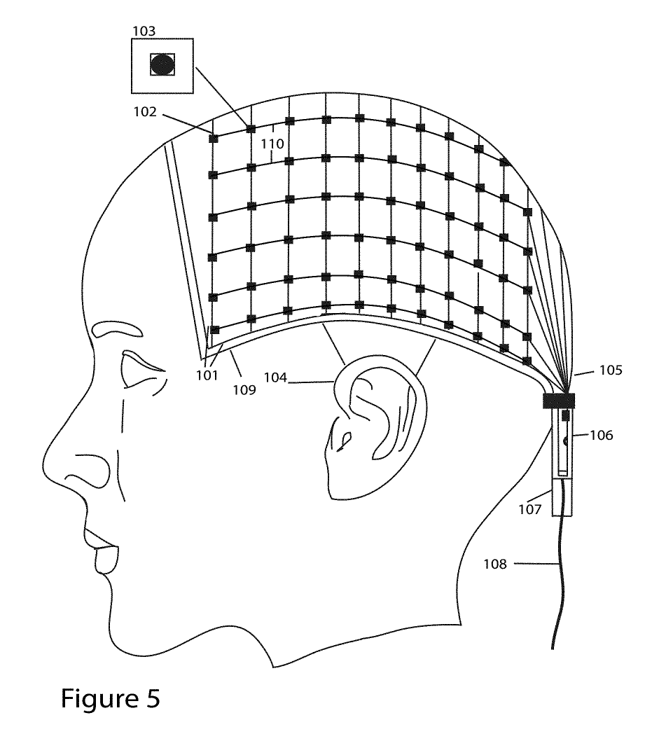

FIG. 5 shows an embodiment of a device with a lower number of electrodes (103). In the version shown in this Figure, there are a total of about 60 to about 100 electrodes. This embodiment is useful when cost is an issue, and/or when the user favors convenience of assembly over data volume and precision. It may also be useful for adapting the device for people of different sizes and age groups.

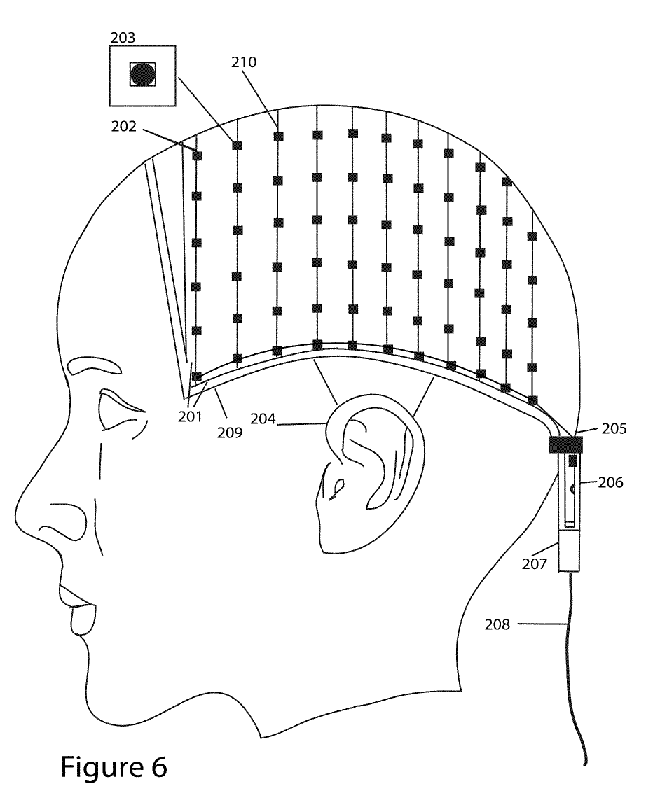

FIG. 6 shows an embodiment of a device with a different wiring pattern. In this case, the wiring pattern forms horizontal lines across the scalp and attaches to the side wires (202), which then connect to the main chip (216). This embodiment may be convenient for those who put on rigid headbands by pulling them partially across their scalp or for those who otherwise wear garments or ornaments that may interfere with vertical wiring.

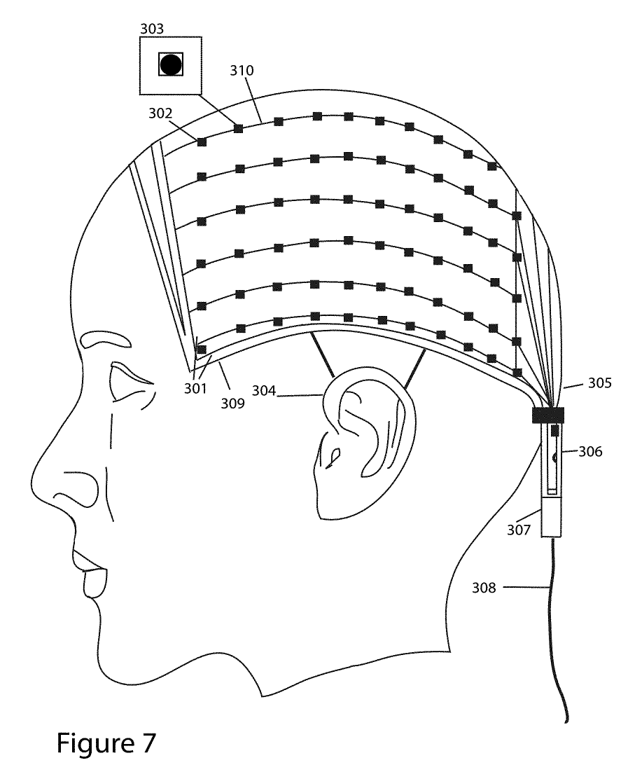

FIG. 7 shows an embodiment of a device (301) with a different wiring pattern. In this case, the wiring pattern involves vertical lines across the scalp connecting to side wires that attach to the main chip. This version of the wiring may allow those with cornrows or braids otherwise patterned along their scalp to conceal the device.

FIG. 8 depicts an examples of a computer program associated with the chip (18). This program's processing has been described above.

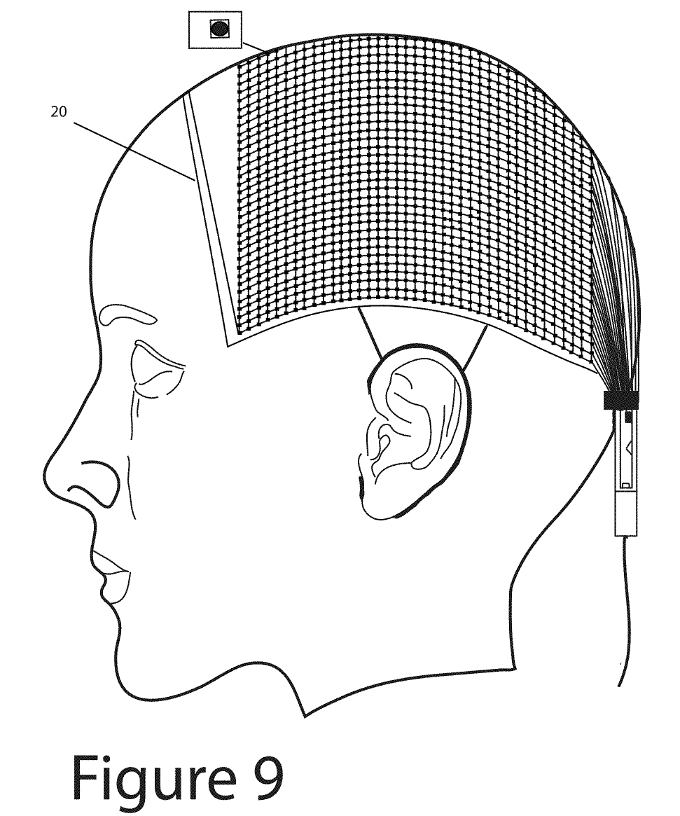

FIG. 9 shows a different elastic pattern that could be used to maximize the comfort of the user (20). This alteration to the system would allow the user to adapt the device to make the device easier to conceal given different hairlines.

FIG. 10 shows embodiments including versions of the device employing olfactory sensors (21), audio recorders (22), and tactile sensors (23) to aid auditory, olfactory, and partial-sensory deficits. Any of these may be waterproofed in the same manner as the camera. Each sensor optionally can be attached to the collar with the same magnet system used on the cameras. The same wire that would otherwise attach to the camera can attach to each of these sensors (1008a-1008c). However, tactile sensors may be positioned elsewhere on the body and connected to the main device with a longer version of the wire as shown in FIG. 10E (23). Each of these wires connects to a similar computer program to the one used for visual image processing. The olfactory program is 10B, the sound processing program is 10D, and the supplementary tactile processing program is 10F. Each of these processing programs modifies the thing measured and unit used appropriately. Creating embodiments like this that relate to different disabilities may allow the device to be adapted in the future to serve the needs of those with other disabilities in a comfortable manner.

The power source described herein is used to power the rest of the system and frequently is attached to the chip (6), which attaches the wires coming from the electrodes or stimuli as well as those from the camera. The disguise feature for the wires is set in place to allow users to use the device without drawing attention away from their face or drawing attention to their impairment. The stimuli or nodes (3) are electrical or tactile to adapt to different cases of visual impairment and levels of scalp sensitivity. The additions of other features described herein allow the device to be adapted to different use cases.

Alternative embodiments of this device vary existing parts to more optimally suit the preferences of particular patients. For example, one embodiment of this device uses cameras with different properties, like heat resistance, size, waterproofing method or 3D capabilities. These adjustments could allow a user to go to saunas, conceal their device more easily, swim, or interact physically in a more comfortable manner with their device. Stimulator nodes in alternative versions of the device are stronger and involve more direct mechanical pressure on the scalp, involve heating or cooling properties, or combinations of these and aforementioned properties. Making these stimulators be similarly resistant would similarly allow users to enjoy their favorite activities without worrying about the welfare of the device. Other adaptations include waterproofing the chip or different parts of the device by encasing them in thermoplastic or thermoset materials such as rubber, HDPE, polyvinyl chloride (PVC), wax, or silicone elastomer. This sort of waterproofing may also be used for the wires and connectors. This further allows parts of the device to be adaptable to those who engage more with water as a hobby. Yet another embodiment of this device may include wires of different sizes, material, or lengths that are insulated or waterproofed with different materials. Making alterations with regards to wire properties may allow users to clean the device more easily if they have poor motor control.

Different attachment methods for the stimulators and the wire grid will further allow the device to be customizable to users preferences. In some cases, these nodes may be implanted to ensure that their electrical or tactile signal is strong enough, or that the device will not come off during physically involving activities. A different way of connecting stimulators to the scalp may also be used in another version of this sensory prosthesis. Examples of other methods for attaching the stimulators to the scalp include partial or full implantation, attaching the nodes to existing hair or permanently gluing the electrodes to the skin on the scalp. Finally, the wire grid may be secured differently, replacing the elastic paths seen in FIGS. 1 through 7 with glue, tape, or nothing at all. Doing so may allow the user to adapt the device to their sensory preferences or any allergy to certain materials.

Alternative versions also employ smaller chips and different programs used to interpret visual information (18), such as programs written in different programming languages or using different ways of compressing the image so the information can be communicated to nodes more quickly. Adapting these aspects of the design will allow it to work more efficiently if the patient performs high-precision tasks on a daily basis. It also allows the device to adapt to new technology.

A number of alternatives, modifications, variations, or improvements therein may be subsequently made by those skilled in the art, which are also intended to be encompassed by the following claims.

What is claimed is:

1. A sensory prosthesis comprising:
a data recording device configured to record data comprising at least one of visual, olfactory, sound, and rerouted tactile data proximate a user,
a plurality of stimulators attached to the user's scalp,
a computer positioned on or proximate to the user, the computer being configured translate the recorded data into signals,
a plurality of wires configured to send signals from the computer to the stimulators, and
a power source configured to power the data recording device and the computer.

2. The sensory prosthesis of claim 1, wherein the data recording device is configured to be removably mounted to the user's clothing.

3. The sensory prosthesis of claim 1, wherein the data recording device is configured to be removably mounted to the user's skin.

4. The sensory prosthesis of claim 1, wherein the plurality of stimulators are waterproofed.

5. The sensory prosthesis of claim 1, wherein the plurality of wires are waterproofed.

6. The sensory prosthesis of claim 1, wherein the plurality of wires are disguised.

7. The sensory prosthesis of claim 1, wherein the plurality of stimulators are tactile or electrical nodes.

8. The sensory prosthesis of claim 1, wherein the plurality of stimulators are microelectrodes.

9. The sensory prosthesis of claim 1, wherein the computer comprises a programmed computer chip.

10. The sensory prosthesis of claim 1, wherein the power source is a portable and rechargeable battery.

11. The sensory prosthesis of claim 1, wherein the data recording device attaches to clothing magnetically.

12. The sensory prosthesis of claim 1, further comprising a wire configured to be hooked over the user's ears to hold wires against the scalp.

13. The sensory prosthesis of claim 1, wherein electrodes are laid against the user's scalp to receive sensory information.

14. The sensory prosthesis of claim 1, wherein the wire structure is made up of separate segments that can be assembled on the user's scalp.

15. The sensory prosthesis of claim 1, wherein the data comprises visual images.

16. The sensory prosthesis of claim 15, wherein the plurality of stimulators are waterproofed.

17. The sensory prosthesis of claim 15, wherein the plurality of wires are waterproofed.

18. The sensory prosthesis of claim 1, wherein the data comprises rerouted tactile information gathered from sensors mounted on at least one of the user's skin and clothing.

19. The sensory prosthesis of claim 1, wherein the data comprises olfactory information gathered from an electronic nose or other olfactory sensor.

20. The sensory prosthesis of claim 1, wherein the data comprises auditory information gathered from an audio recording device.
